(19) United States
(12) Patent Application Publication
(10) Pub. No.: US 2025/0099173 A1
(43) Pub. Date: Mar. 27, 2025

(54) BLOOD VESSEL VASCULAR TONE MODULATING DEVICE

(71) Applicant: NEOFLOW TECHNOLOGIES LTD, Nazareth (IL)

(72) Inventor: David SKOROHOD, Netanya (IL)

(21) Appl. No.: 18/970,927

(22) Filed: Dec. 6, 2024

Related U.S. Application Data
(63) Continuation-in-part of application No. PCT/IB2023/055825, filed on Jun. 6, 2023.
(60) Provisional application No. 63/728,203, filed on Dec. 5, 2024.
(60) Provisional application No. 63/350,060, filed on Jun. 8, 2022.

(51) Int. Cl. A61B 18/14; A61B 18/00; A61B 18/12

(52) U.S. Cl. CPC A61B 18/1492; A61B 2018/00083; A61B 2018/0022; A61B 2018/00404; A61B 2018/1226; A61B 2018/1435

(57) ABSTRACT

A vascular tone modulating device for controlling the tension and diameter of blood vessels includes modulating member, a supporting structure, a stimulation circuit and a conductive pathway. The modulating member includes at least one electrode and the supporting structure supports the modulating member within a blood vessel. The conductive pathway extends along the supporting structure and electrically connects the stimulation circuit and the modulating member and the vascular tone modulating device is of a stand-alone configuration.

### TECHNICAL FIELD

Embodiments of the present disclosure generally relate to neuro, cardiology, and/or peripheral blood vessel vascular tone modulating devices.

### BACKGROUND

Vascular spasm is a reaction of the smooth muscle cells of the blood vessel to different stimulations like temperature change, injury, inefficient metabolism, trauma, intoxication, and possible contact of a catheter with an inner lumen of a blood vessel.

Catheter-induced spasm is a known complication during catheterization procedures which can cause a delay during the intervention procedure and may cause blood clot formation in cerebral or other procedures.

Vasospasm occurring during medical treatment due to e.g. traumatic and/or other possible disease related events, may cause flow limitation and subsequent difficulty with diagnosis or treatments.

A common practice of dealing with the vasospasm phenomena in clinical intervention may be use (in separate or in combinations) of: intra-arterial lidocaine injection, intra-arterial nicardipine injection, warm compress, angioplasty balloons (and the like).

### SUMMARY

Aspects of the disclosure, according to some embodiments thereof, relate to vascular tone modulating devices, such as a spasm relief device. More specifically, but not exclusively, aspects of the disclosure, according to some embodiments thereof, relate to spasm relief device configured for pass obstacle of the blood vessel closure generated by spasm by inducing electrical signal to the spasmatic blood vessel target.

The spasm relief device and methods of the present disclosure allow for recanalization of the spasmatic difficult-to-pass blood vessels to minimize damage to surrounding blood vessel tissue. Advantageously, a spasm relief device of the present disclosure uses electric impulses to stimulate the endothelial layer for releasing nitric oxide (NO) molecules to the smooth muscle of the blood vessel to activate the relaxation mechanism. By that, avoidance and/or reduction of the use of medications or angioplasty devices or dilating devices may be possible, hence reducing the risk of perforation or dissection of the blood vessel wall.

According to an aspect of some embodiments, there is provided an invention for blood vessel spasm relaxation by a device comprising a proximal hub, device shaft, and a tip. The energy and a regulating circuit located at a proximal hub may activate the device to function by a user applying minor pressure to the button located on the connecting hub. The control button may activate the regulation circuit to induce an electric current to the target tissue.

The impulse introduction may be possible by monopolar or bipolar effect. A cathode or an anode set of each separate electrode can be controlled by the operation mode regardless of the recent activation. An operation mode can be selected by the operator by pressing the power button or by a programmed sequence. The electric regulation circuit may be capable of discharging in certain cases the current in the range of about 0.1 micro-Ampere to about 100 milli-Ampere.

The electrodes may be exposed in the operating section of the device, mainly the distal end. The number of electrodes may vary from 1 to about 100 units. The electrodes distribution over the tip and the shaft may vary between or in the combination of the axial and the radial pattern. The power bank of the spoken invention may house a sufficient amount of energy to accomplish the clinical intervention, for example a total energy capacity of up to about 1000 micro-Ah. The power bank may be configured to provide an electric potential of up to about 75 v in certain cases. A hollow member may be designed to deliver the generated impulse to the device tip through conductive elements contained in the shaft. The shaft may be fabricated from a combination of metal and plastic, or any equivalent used in the field of medical device fabrication methods and standards.

The shaft may be designed for flexibly to allow navigation traceability and pushabilty in the blood vessel anatomy in a safe manner. The diameter of the shaft can vary in the range of about 0.1 mm to about 30 mm parallel or tapered. The length can vary in the range of about 3 cm to about 300 cm. The tip may include in certain cases exposed electrodes able to induce the generated signal to the spasmatic tissue, while the atraumatic tip may be designed for advancement and retraction in the blood vessel without substantially compromising the safety and avoiding the blood vessel wall from perforation and dissection.

The previously described spasm relief devices accordingly represent one example of a vascular tone modulating device as outlined in this disclosure. Another example of such a device could be a vasoconstriction device. Typically, a vascular tone modulating device designed for vasoconstriction shares similar technical and structural features with the spasm relief devices described herein. However, it primarily differs in its intended effect of inducing the narrowing of blood vessels rather than their relaxation and widening, through the application of specific electric impulses that stimulate such blood vessels to narrow. Further aspects of the present invention are exemplified in the following with respect to a vascular tone modulating device in form of a spasm relief device:

1. A blood vessel spasm relief device for pass obstacle of the blood vessel closure, the blood vessel spasm relief device comprising:
a connecting hub facilitating push-button mechanism, power bank, and an electric circuit configured for selective power transfer;
a strain relief, prolonged flexible member, configured for the gradual spreading of the bending moment applied on the joint of the hollow member-connecting hub to avoid hollow member kink;
a hollow member, which is elongated and comprises a main section and a distal section, configured to transfer the electric current to the tip;
a tip, the distal end of the hollow member facilitating exposed electrodes wired to the electrical circuit located in the connecting hub configured for atraumatic device advance and retraction in the blood vessel lumen and introduction of the electric current to the contacting and near blood vessel tissue;

wherein the tip is configured to relief blood vessel spasm by inducing electric current, based on the intervention need when affecting the smooth muscle cell by an electro-stimulating endothelial layer of the blood vessel activating the secretion of NO molecule causing smooth muscle cell relaxation.

2. A blood vessel spasm relief device for pass obstacle of the blood vessel closure, the blood vessel spasm relief device comprising:
a connecting hub facilitating push-button mechanism, power bank, and an electric circuit configured for selective power transfer;
a prolonged member, which is constructed with solid material or or coaxial assembly of at least 2 members or tangentially attached members elongated and comprises a main section and a distal section, configured to transfer the electric current to the tip;
a tip, the distal end of the prolonged member facilitating exposed electrodes wired to the electrical circuit located in the connecting hub configured for atraumatic device advance and retraction in the blood vessel lumen and introduction of the electric current to the contacting and near blood vessel tissue;

wherein the tip is configured to relief blood vessel spasm by inducing electric current, based on the intervention need when affecting the smooth muscle cell by an electro-stimulating endothelial layer of the blood vessel activating the secretion of NO molecule causing smooth muscle cell relaxation.

3. A blood vessel spasm relief device for pass obstacle of the blood vessel closure, the blood vessel spasm relief device comprising:
a connecting hub facilitating push-button mechanism, power bank, and an electric circuit configured for selective power transfer;
a strain relief, prolonged flexible member, configured for the gradual spreading of the bending moment applied on the joint of the hollow member-connecting hub to avoid hollow member kink;
a hollow member, which is single lumen or multi-lumen or partially multi-lumen or coaxial elongated and comprises a main section and a distal section, configured to transfer the electric current to the tip;
a balloon applied over distal segment of the hollow member, which is compliant or semi-compliant or non compliant and inflated with liquid or gas and covered with conductive metal or plastic material and exposed electrodes
a tip, the distal end of the hollow member facilitating exposed electrodes wired to the electrical circuit located in the connecting hub configured for atraumatic device advance and retraction in the blood vessel lumen and introduction of the electric current to the contacting and near blood vessel tissue;

wherein the tip is configured to relief blood vessel spasm by inducing electric current, based on the intervention need when affecting the smooth muscle cell by an electro-stimulating endothelial layer of the blood vessel activating the secretion of NO molecule causing smooth muscle cell relaxation.

4. The blood vessel spasm relief device of aspects 1-3, wherein the hollow member is made of metal and plastic for needed flexibility for effective blood vessel navigation, including axial conductive material allowing translation of an electric signal from hub to devise tip

5. The blood vessel spasm relief device of aspect 4, wherein the hollow member diameter can be in the range of 0.1 mm to 30 mm parallel or tapered, and the length can be in the range of 3 cm to 300 cm.

6. The blood vessel spasm relief device of aspect 4, wherein the tip distally projects from a distal end of the distal section.

7. The blood vessel spasm relief device of aspects 1-6, wherein the number of the exposed electrodes is at least one.

8. The blood vessel spasm relief device of aspect 7, wherein the exposed electrodes are distributed axially or radially or in combination.

9. The blood vessel spasm relief device of aspect 8, wherein the electric circuit regulates the single, paired, and grouped discharge through the electrodes regardless of the last operation.

10. The blood vessel spasm relief device of aspect 8, wherein the electric circuit regulates the polarization cathode or anode of each electrode regardless of the recent operation.

11. The blood vessel spasm relief device of aspects 8-10, wherein the polarization and single or pairing or group discharge function can regulate by push button or programmed sequence.

12. The blood vessel spasm relief device of aspect 11, wherein the power button facilitates the ergonomic design and allows the user to selectively and on-demand induce electric signal to the device tip.

13. The blood vessel spasm relief device of aspect 11, wherein the power bank is attached to the electric circuit and stores a sufficient amount of energy to complete the intervention procedure while used in an adequate manner.

14. The blood vessel spasm relief device of aspect 11, wherein the power bank is providing the electric potential of up to 5 v.

15. The blood vessel spasm relief device of aspect 11, wherein the power bank stores a total amount of electric energy of up to 1000 mAh.

16. The blood vessel spasm relief device of aspect 11, wherein the electric circuit, is supplied by the energy of the power bank and activated by the power button, is wired to the tip.

17. The blood vessel spasm relief device of aspect 16, wherein electric circuit regulates the discharge current in the range of 0.1 micro-Ampere to 100 milli-Ampere.

18. The blood vessel spasm relief device of aspect 16, wherein the distal end of the hollow member translates to tip, facilitates the atraumatic distal end allowing the progressing and retraction in the blood vessel anatomy in a safe manner without perforating or dissecting the blood vessel wall. Atraumatic, distal end diameter can be in the range of 0.1 mm to 30 mm parallel or tapered.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the disclosure are described herein with reference to the accompanying figures. Together with the figures, the description makes apparent to a person having ordinary skill in the art how some embodiments may be practiced. The figures are for the purpose of illustrative description, and no attempt is made to show structural details of an embodiment in more detail than is necessary for a fundamental understanding of the disclosure. For the sake of clarity, some objects depicted in the figures are not to scale.

### In the Figures:

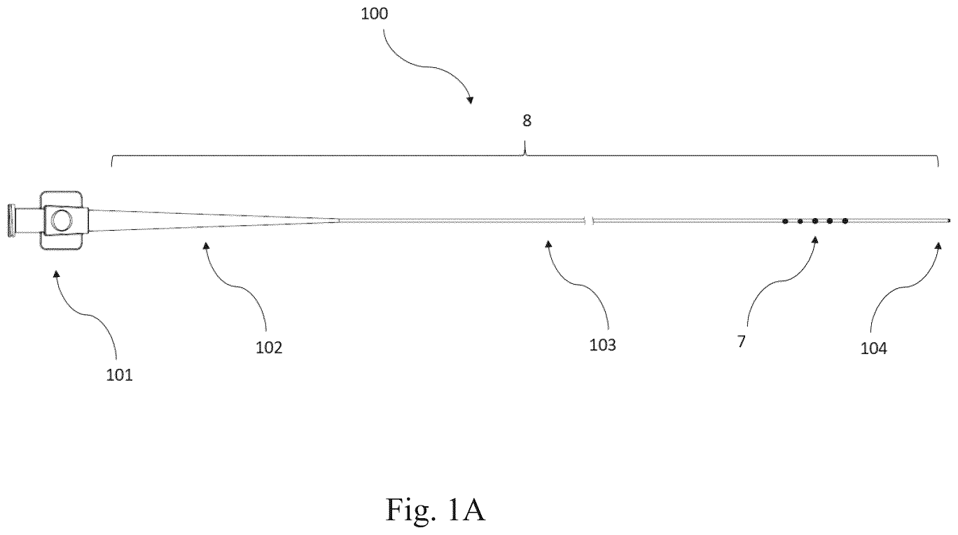

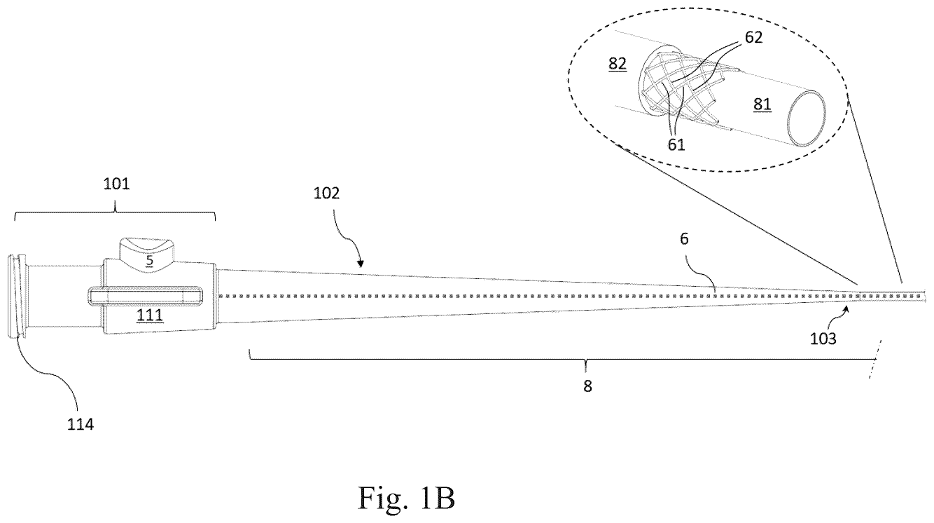

FIG. 1A is a schematic, top view of a blood vessel vascular tone modulating device configured for controlling the tension and diameter of a blood vessel, according to some embodiments;

FIG. 1B is a schematic, side view of the proximal end of the vascular tone modulating device of FIG. 1A, according to some embodiments;

FIG. 1C is a schematic, side view of the distal end of the vascular tone modulating device of FIG. 1A, according to some embodiments;

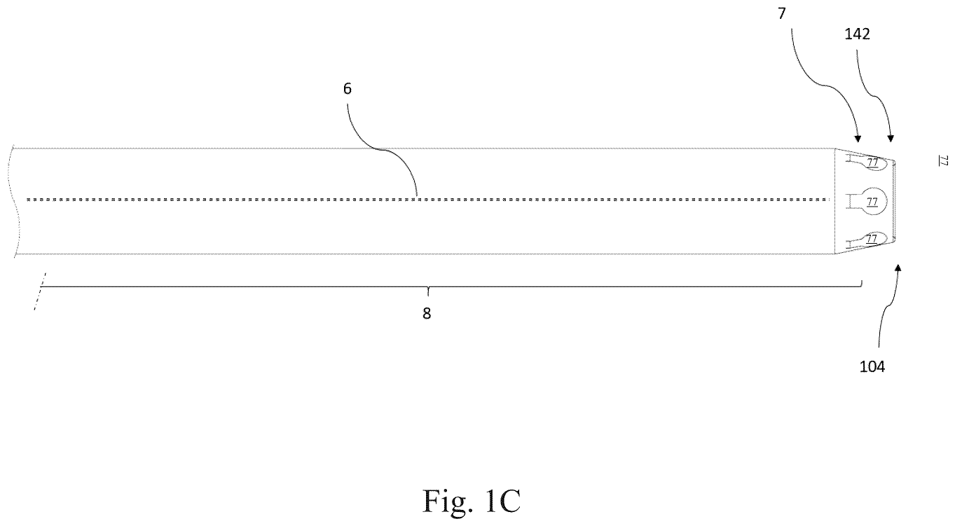

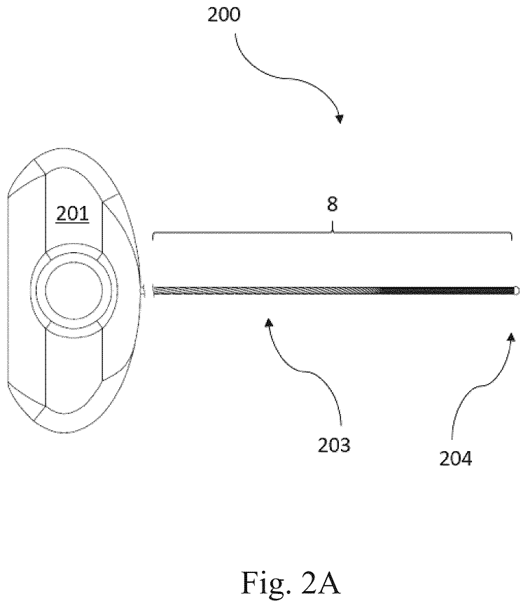

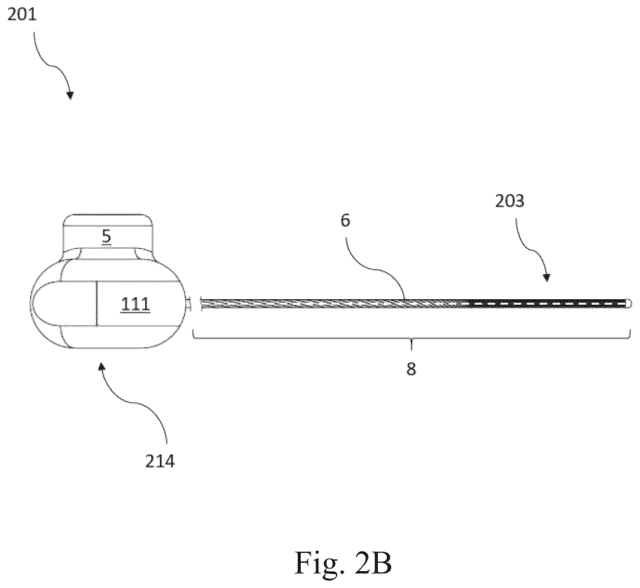

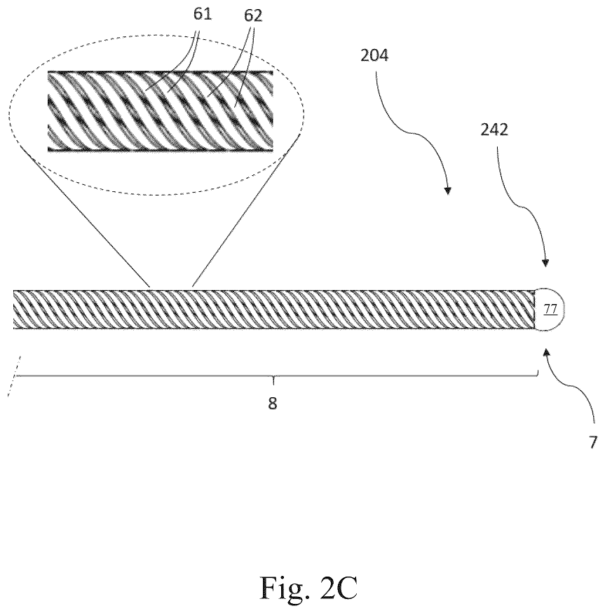

FIG. 2A is a schematic, top view of a blood vessel vascular tone modulating device configured for controlling the tension and diameter of a blood vessel, according to some embodiments;

FIG. 2B is a schematic, side view of the proximal end of the vascular tone modulating device of FIG. 2A, according to some embodiments;

FIG. 2C is a schematic, side view of the distal end of the vascular tone modulating device of FIG. 2A, according to some embodiments;

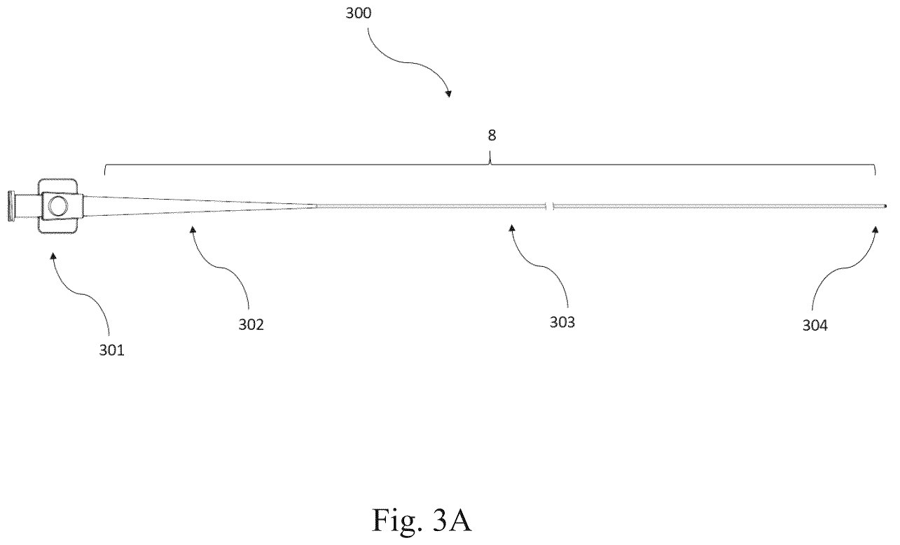

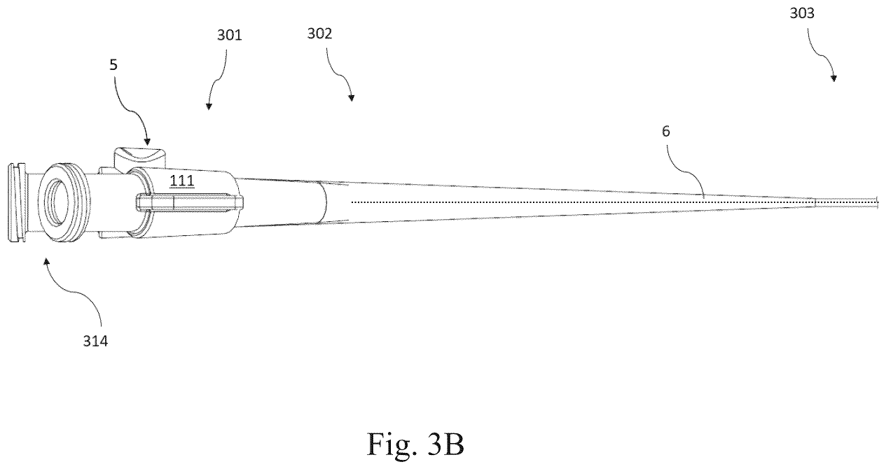

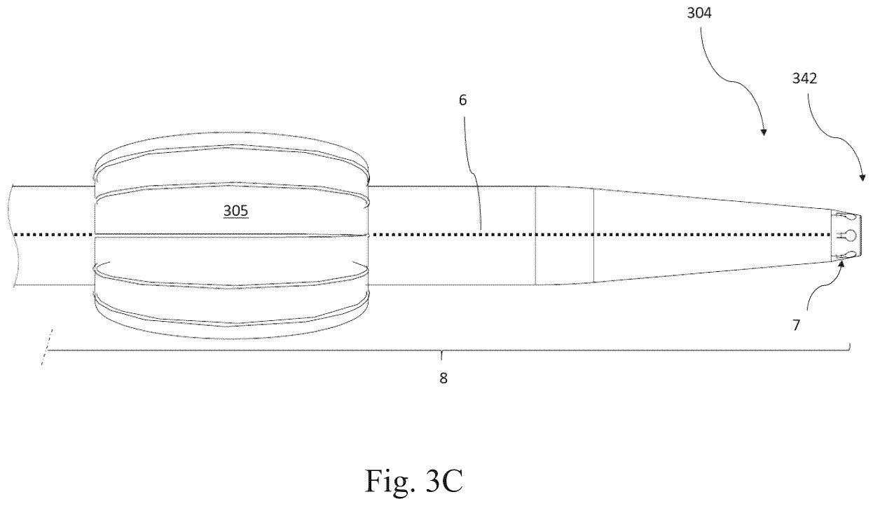

FIG. 3A is a schematic, top view of a blood vessel vascular tone modulating device configured for controlling the tension and diameter of a blood vessel, according to some embodiments;

FIG. 3B is a schematic, side view of the proximal end of the vascular tone modulating device of FIG. 3A, according to some embodiments;

FIG. 3C is a schematic, side view of the distal end of the vascular tone modulating device of FIG. 3A, according to some embodiments;

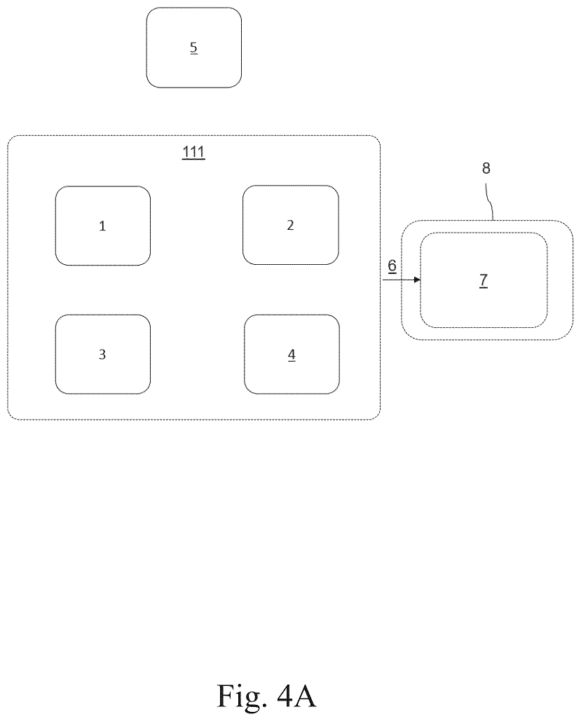

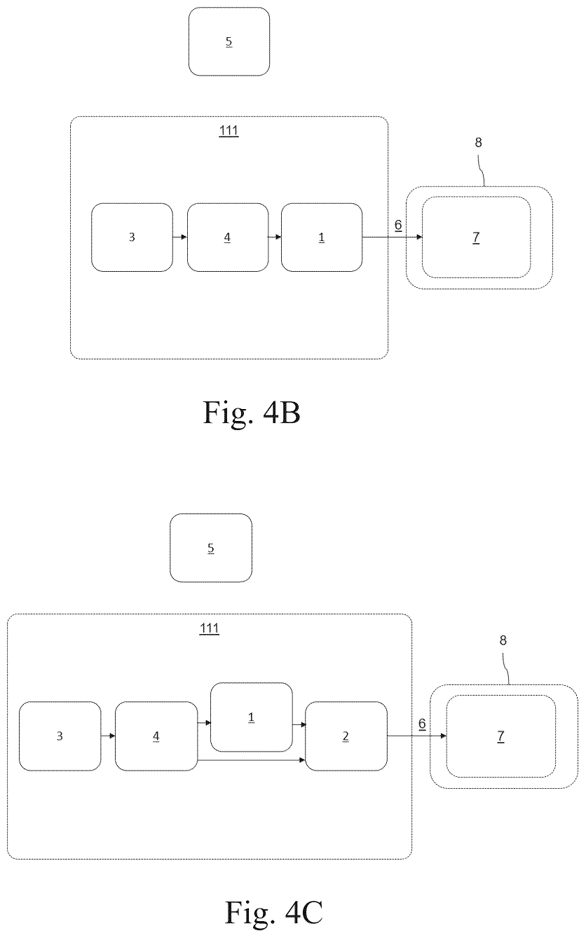

FIGS. 4A to 4C are schematic views of stimulation circuits of certain vascular tone modulating device embodiments of the invention;

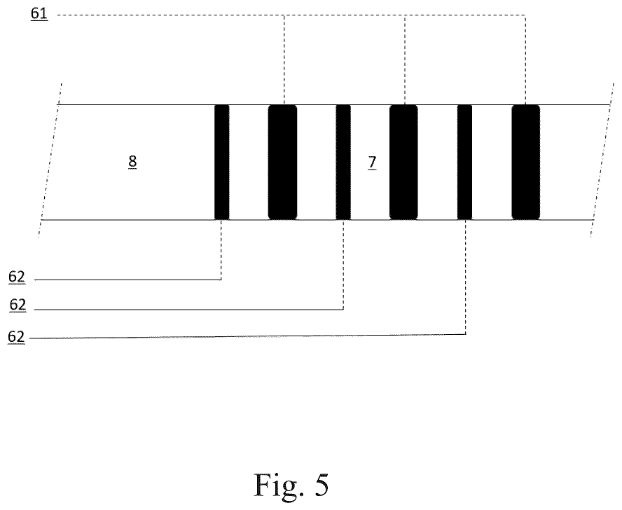

FIG. 5 is a schematic view of a modulating member and conductive pathway of a vascular tone modulating device in accordance with various embodiment of the invention;

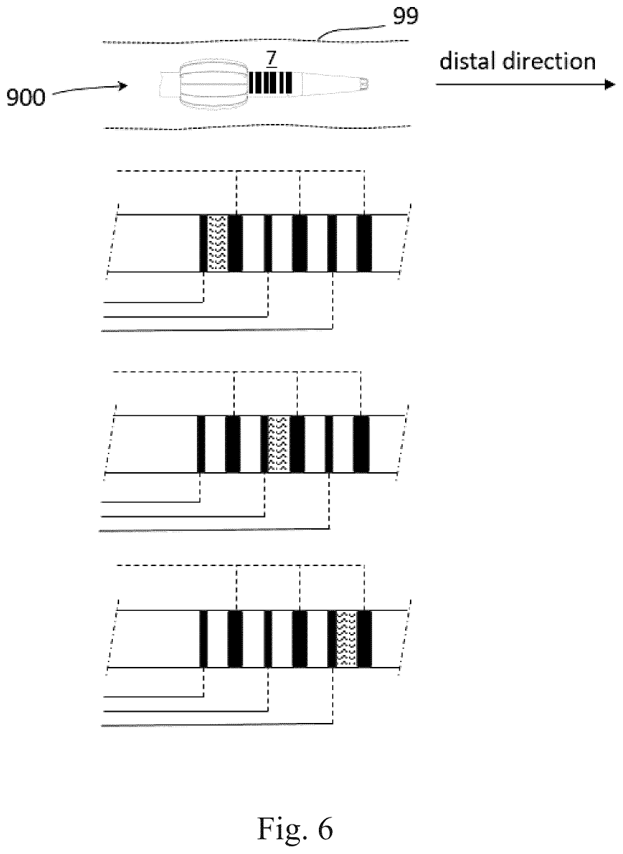

FIG. 6 is a schematic view showing a possible use of a modulating member and conductive pathway generally similar to that of FIG. 5 in forming a wave impulse for enhancing tone modulating in a blood vessel;

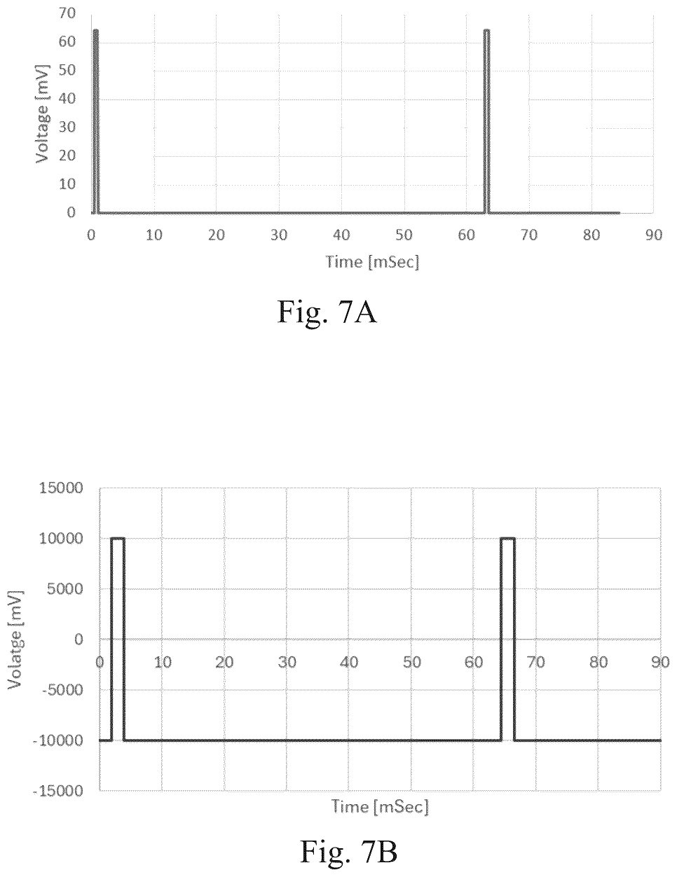

FIGS. 7A and 7B are schematic views showing examples of wave impulses that can be created by at least certain vascular tone modulating device embodiments of the invention;

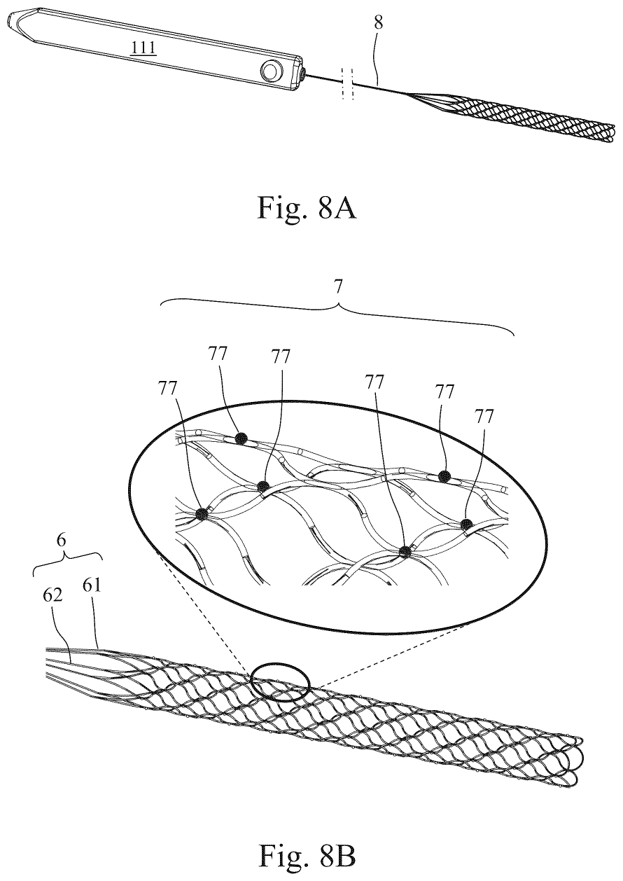

FIGS. 8 to 10 are schematic views of exemplary conventional interventional devices or instruments that may be implemented to include various aspects of the vascular tone modulating device embodiments of the invention;

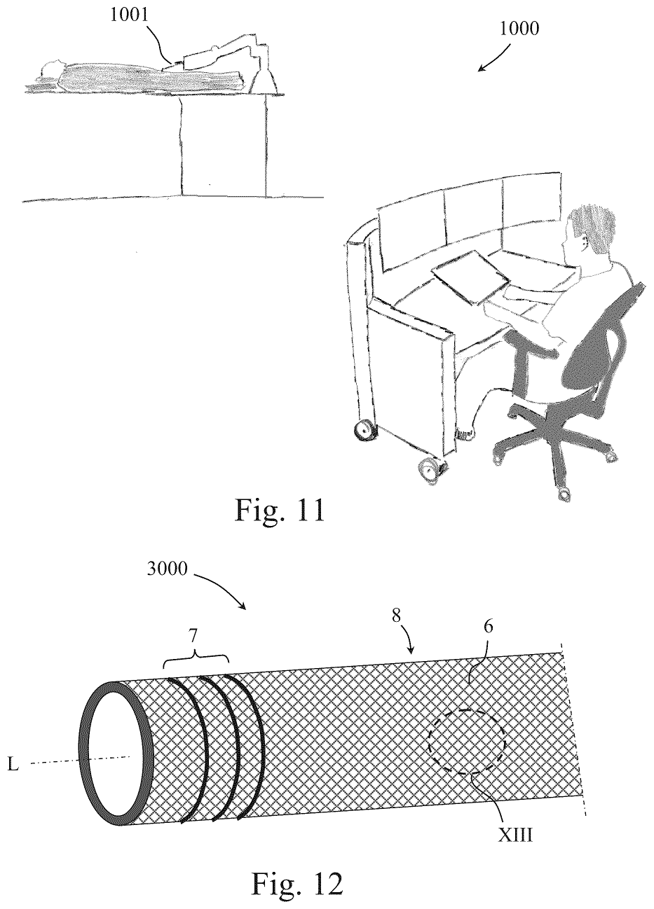

FIG. 11 schematically shows an endovascular robotic system for performing minimally invasive procedures in which embodiments of vascular tone modulating devices of the present disclosure may be utilized.

FIG. 12 schematically shows an interventional device that may be implemented as a vascular tone modulating device in accordance with an embodiment of the present invention;

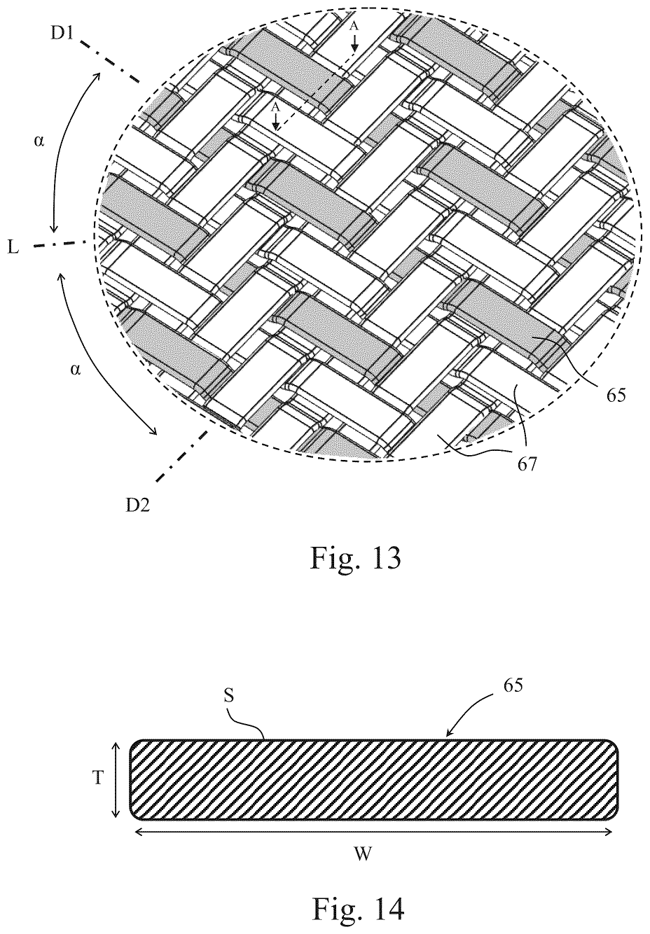

FIG. 13 provides an enlarged view of section XIII marked in FIG. 12, showing a conductive pathway as a braided structural element;

FIG. 14 schematically shows a view taken along plane A-A indicated in FIG. 13, showing a cross sectional view of a conductive thread in accordance with an embodiment of the present invention;

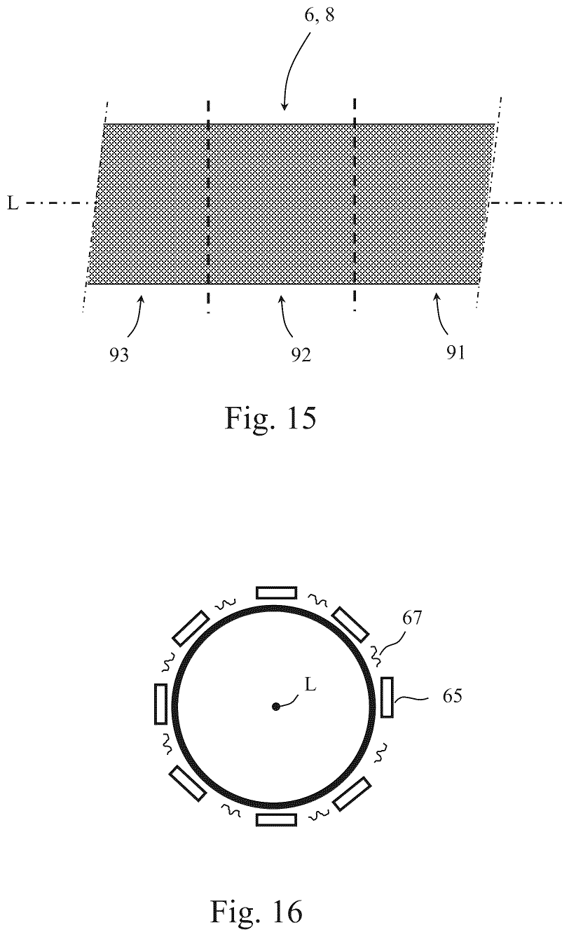

FIG. 15 schematically shows a section of a conductive pathway of an interventional device's supporting structure in accordance with an embodiment of the present invention;

FIG. 16 schematically shows a cross sectional view of the interventional device seen in FIG. 12, taken along a plane perpendicular to its longitudinal axis; and

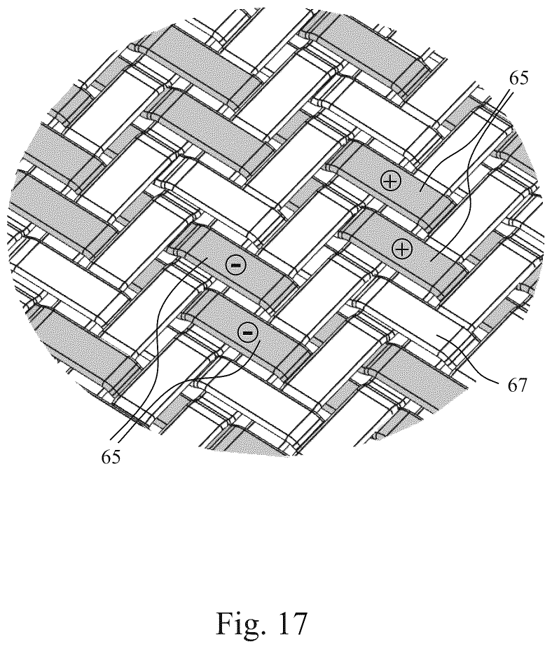

FIG. 17 schematically shows a view generally similar to FIG. 13 of another embodiment of a conductive pathway formed as a braided structural element.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, the same reference numerals refer to the same parts throughout.

In the description and claims of the application, the words “include” and “have”, and forms thereof, are not limited to members in a list with which the words may be associated.

In the description and claims of the application, the expression “at least one of A and B”, (e.g, wherein A and B are elements, method steps, claim limitations, etc.) is equivalent to “only A, only B, or both A and B”. In particular, the expressions “at least one of A and B”, “at least one of A or B”, “one or more of A and B”, and “one or more of A or B” are interchangeable.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. For example, the statement “the length of the element is equal to about 1 m” is equivalent to the statement “the length of the element is between 0.8 m and 1.2 m”. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “substantially” and “about” may be interchangeable.

For ease of description, in some of the figures, a three-dimensional cartesian coordinate system (with orthogonal axes x, y, and z) is introduced. It is noted that the orientation of the coordinate system relative to a depicted object may vary from one figure to another. Further, the symbol ⊙ may be used to represent an axis pointing “out of the page”, while the symbol ⊗ may be used to represent an axis pointing “into the page”.

Embodiments of the present disclosure relate to electrical stimulation that can be applied directly e.g. to specific nerves that control blood vessel dilation. This can be done through invasive procedures, such as implanting electrodes near the targeted nerves and by delivering electrical impulses to these nerves in order to promote e.g. the smooth muscle cells in the blood vessel walls to relax, leading to vasodilation.

FIG. 7A provides an optional example of a square wave impulse that can be used for enhancing vasodilation in accordance with various embodiments of the present invention. This square wave has an amplitude of about 64 millivolts, a frequency of about 16 Hz, a pulse width of about 2 milliseconds, and may be produced by an embodiment of a vascular tone modulating device in form of a spasm relief device having a distance between its electrodes of about 10 millimeters.

FIG. 7B provides an optional example of a square wave impulse that can be used for enhancing vasoconstriction in accordance with various embodiments of the present invention. This square wave has an amplitude of about 20,000 millivolts peak to peak, a frequency of about 16 Hz, a pulse width of about 2 milliseconds, and may be produced by an embodiment of a vascular tone modulating device in form of a spasm relief device having a distance between its electrodes of about 10 millimeters.

Typically, vascular tone modulating devices that enhance vasodilation or vasoconstriction share similar technical and structural features and primarily differ in their intended effects of widening or narrowing of blood vessels, respectively. Such desired intended effects may be achieved through the application of specific electric impulses that stimulate the blood vessels. Accordingly, FIG. 7A provides an example of such a wave impulse for enhancing vasodilation, while FIG. 7B provides an example of such a wave impulse for enhancing vasoconstriction.

By way of a non-binding example, a wave impulse having an amplitude of up to about 1000 millivolts may be useful for enhancing vasodilation and a wave impulse having an amplitude of between about 5000 and about 50,000 millivolts (and possibly greater) may be useful for enhancing vasoconstriction.

In one broad aspect of the present disclosure, at least certain vascular tone modulating device embodiments of the present invention, such as those exemplified herein—may be of a stand-alone configuration. That is to say, that such stand-alone device embodiments may be designed to be capable of operating for their intended use of blood vessel tone modulating, in an independent manner substantially without the need for external connections or additional equipment. In other words, such stand-alone configuration may be defined as being substantially self-contained and designed to perform its intended functions substantially without relying on other external devices or systems.

With attention drawn to FIG. 4A, an embodiment of a stimulation circuit 111 that can be suitable for facilitating such stand-alone mode of operation of a blood vessel vascular tone modulating device—may be seen including the following components: a pulse generator 1, an optional amplifier 2, a battery 3 and a power supply 4. A power button 5 provided on such a stand-alone device may be used for activating and powering the stimulation circuit 111.

FIGS. 4B and 4C illustrate various embodiments of stimulation circuits 111.

In FIG. 4B, an embodiment of a stimulation circuit 111 having a battery 3, a power supply 4 and a pulse generator 1 is shown. In this example, the battery 3 provides an input voltage Vin to the power supply 4, which in turn outputs an output voltage Vs towards the pulse generator.

In FIG. 4C, an embodiment of a stimulation circuit 111 that also includes an optional amplifier 2 is shown. Here the battery 3 provides an input voltage Vin to the power supply 4, which in turn outputs output voltages Vs towards the pulse generator and the amplifier 2.

The amplifier 2 in various embodiments may be any one of the following: a voltage amplifier, a current amplifier, or a combined voltage and current amplifier. By using an amplifier 2, the outputted wave impulse delivered towards a targeted area within the body where vasodilation is promoted, may have a higher voltage amplitude and/or power than otherwise would be provided from the signal generator alone.

In non-binding examples: Pulse Generator 1 may be the LMC555 or MIC1555/57 versions of 555 timer integrated circuit manufactured by Texas Instruments; Amplifier 2 may be the LM4916 or LM2621MM/NOPB power amplifiers manufactured by Texas Instruments; Battery 3 may the SR421SW model of silver oxide button cell battery of Seiko Instruments Inc.; Power Supply 4 may be the TPS61098 Low input voltage, 4.3-V output voltage, synchronous boost converter with integrated LDO; and the power button 5 may be used for activating/deactivating a TL3305 Series ultraminiature, SMT tactile switch of E-Switch, Inc. a wholly owned subsidiary of Stein Industries, Inc.

A blood vessel vascular tone modulating device in form of a spasm relief device including such a stimulation circuit 111—may be arranged to transmit electrical signals along a conductive pathway 6 towards a modulating member 7 of the device comprising one or more electrodes, which may be supported by a supporting structure 8 of the device within blood vessels (see blood vessel 99 in FIG. 6) experiencing vasoconstriction.

Stimulating the modulating member 7 may accordingly result in vasodilation of such blood vessels, by widening or relaxing the blood vessels, leading to an increase in their diameter-which in turn promotes increased blood flow and decreased vascular resistance.

In a broad aspect of the present invention, conductive pathways 6 of various embodiments of blood vessel vascular tone modulating devices, in addition to functionally serving as a transmission pathway for electrical signals back and forth towards the modulating member 7, may also be configured to functionally serve as a structural component for enhancing structural stability of the device and in particular it's supporting structure 8.

In certain embodiments, such bi-functionality may be accomplished by forming a conductive pathway 6 as a braided structural element, where one or more electrical conducting wires may be interlaced or intertwined together, possibly together with other non-conductive strands.

Such braiding may be in any one of the following ways: one wire/thread over two-under two; two wires/threads over two-under two; one wire/thread over one-under one.

Possibly such a braided conductive pathway 6 may be encased within insulating material in order to avoid substantial interference to other components, such as electrodes, of the device. In addition or alternatively, each conductive wire within the braid may be encased within insulating material.

In the enlarged sections provided in FIGS. 1B and 2C, two examples of such bi-functional conductive pathways 6 are shown. In the enlarged section of FIG. 1B, the conductive pathway is shown having interlaced or intertwined conducting wires 61, 62 of opposite poles.

This braided conductive pathway is here seen placed in a peripheral formation between inner and outer layers 81, 82 of the supporting structure 8, where possibly these layers may be of insulating material. Inner layer 81 may form an outer periphery of an internal lumen of the micro catheter type interventional device seen in FIG. 1.

With attention drawn to FIG. 5, a schematic view is provided exemplifying various ways in which electrodes of a modulating member 7 may be electrically connected to electrical wires of a conductive pathway 6 (braided or not).

In this figure, a supporting structure 8 of a blood vessel vascular tone modulating device is shown, including a modulating member 7 having optionally six electrodes. For illustration purposes only, the electrodes are presented in different widths to assist in distinguishing between different sets of electrodes according to their use in the device (i.e. as anodes or cathodes).

In this example, the electrical conducting wires of the conducting pathway are shown using ‘dashed’ lines, and as seen one single wire 61 acting as a “common” (of either the ‘plus’ or ‘minus’ pole) is connected to all the electrodes of one of the electrode sets (i.e. the “wider” ones), while each electrode of the other set (i.e. the “thinner” ones) in this example is connected to a dedicated wire 62 of the opposite pole.

In this way, a total of four electrical wires can be used in the conductive pathway 6 for connecting between the stimulation circuit 111 and the modulating member 7.

In certain embodiments, the minimal number of conductor lines of the conducting pathway may be calculated according to the following formula:

$$minimal Number of conductors = \frac{Number of electrodes}{2} + 1$$

Attention is drawn to FIG. 6 illustrating an optional example of providing a wave impulse to enhance tone modulating in a blood vessel by using an embodiment of a blood vessel tone modulating device having a modulating member 7 and a conductive pathway 6 generally similar to the one seen in FIG. 5.

The upper side of FIG. 6 shows an example of a distal region of a blood vessel tone modulating device 900, such as of the optional design seen in FIGS. 3, being located within a blood vessel 99—while the three illustrations below show the modulating member 7 and conductive pathway 6 of FIG. 5 during three phases of activation.

The single wire 61 (either of positive or negative pole) is accordingly here connected to all the electrodes of the “wider” ‘set’ of electrodes, while a dedicated wire 62 of a given opposite pole is connected to each one of the electrodes of the second ‘set’ of “thinner” electrodes.

In accordance with certain embodiments of the invention, by selectively controlling only one of the dedicated wires 62 to be connected each time to the given pole of the device's power supply, each time only one pair of electrodes can be controlled to create a wave impulse at a given axial location along the blood vessel (as seen by the moving “wavy patterns”).

As illustrated from top to bottom in FIG. 6, an optional example of creating a wave impulse that starts at a more proximal side of this section of the blood vessel and gradually moves in the distal direction is seen. Such a distally moving wave form may assist in certain cases in propagation of tone modulation of a blood vessel in its distal (possible downstream) direction.

FIG. 1A schematically depicts one optional example of an embodiment of a blood vessel tone modulating device 100 for enhancing tone modulation of blood vessels. Blood vessel tone modulating device 100 includes a proximal connecting hub 101 and a supporting structure 8 in this example in the optional form of a strain relief section 102 and a hollow member 103, which is elongated and connected to the hub 101.

The proximal end of the hollow member 103 in this example may be seen optionally supported via the strain relief 102 to the hub in order to provide flexibility that facilitates bending between hollow member 103 and hub 101 in order to avoid or limit kinks.

The distal end of the hollow member 103 extends towards a tip 104 of the device that may be configured to provide an atraumatic distal end for the device. According to some embodiments, hollow member 103 may be made of metal and/or plastic for flexibility and effective blood vessel navigation.

With attention additionally drawn to FIG. 1B, hub 101 can be seen including a catheter connection interface 114 and a stimulation circuit 111 connected and/or embedded within the hub. It is noted that although here seen being associated with the hub, other locations within the device may be equally possible in the various embodiments of the present invention for housing stimulation circuit 111.

Coupling (e.g. embedding) stimulation circuit 111 with the hub may enable an embodiment of blood vessel tone modulating device 100 to function in a stand-alone mode. In other embodiments (not shown), the stimulation circuit 111 may be external to the device hence resulting in an embodiment of blood vessel spasm relief device that is non-stand-alone.

As seen in FIG. 1B, the blood vessel tone modulating device 100 includes a conductive pathway 6 that extends along the device's supporting structure 8 for conducting electric signals from the hub 101 towards a modulating member 7 that may be located along the supporting structure, and possibly also adjacent tip 104.

As disclosed herein, modulating member 7 may be embodied as a plurality of electrodes (see, e.g., electrodes 77 in FIG. 1C), while the conductive pathway 6 may be suited to conduct electrical signals towards one or more of the electrodes of the modulating member, possibly without substantial interference to other electrodes of the device.

According to some embodiments, a diameter of hollow member 103 can be in the range of about 0.3 mm to about 30 mm and may extend generally in a parallel or a tapered formation. A length of hollow member 103 can be in the range of about 3 cm to about 300 cm.

As seen in FIG. 1B, a power button 5 may be included in this example in the hub thus facilitating an ergonomic design and allowing a user of the device to selectively and on-demand activate components of the stimulation circuit 111 to induce electric signals towards modulating member 7. Battery 3 that is included in the stimulation circuit 111 may be suited for storing a sufficient amount of energy to complete a tone modulating procedure.

In one example, battery 3 may provide an electric potential of up to about 5 v and may store a total amount of electric energy of up to about 1000 mAh. Components of stimulation circuit 111, powered by the battery 3 and activated by button 5, may be arranged to direct electric current to electrodes in the modulating member 7 to each electrode separately, to pairs of electrodes, or to groups of electrodes. A type of the tone modulating action to be activated can be selected by button 5 or by a programmed sequence. Stimulation circuit 111 in certain cases may be arranged to regulate the discharge current in a range of about 0.1 micro-Ampere to about 100 milli-Ampere.

FIG. 1C is a schematic, side view of the distal end of the tone modulating device of FIG. 1A, according to some embodiments. The distal end of the hollow member 103 translates to tip 104, accordingly facilitating an atraumatic distal end 142 allowing the progressing in the blood vessel anatomy in a safe manner without perforating or dissecting the blood vessel wall. Atraumatic distal end 142 diameter can be in the range of about 0.3 mm to about 30 mm parallel or tapered.

In this example, modulating member 7 includes electrodes 77 exposed at the tip 104 portion to allow inducing electric current to the near or contact tissue. The number of exposed electrodes on the hollow member 103 and the tip 104 can be in the range of 1 unit to 100 units. The electrodes 77 can be distributed axially or radially (as here seen) or in any such combination the supporting structure 8.

Blood vessel tone modulating device 100 as seen in FIGS. 1A to 1C is embodied upon a so called micro catheter type interventional device, however as will be seen in the following discuses embodiments, blood vessel tone modulating device embodiments of the present disclosure may be implemented in a variety of other conventional interventional devices or instruments, such as guide-wires, balloon catheters, stent retrievers (etc.). In addition, blood vessel tone modulating device embodiments may also be implanted in dedicated interventional devices tailored for tone modulating and not necessarily only in existing types of interventional devices.

With attention drawn to FIGS. 8 to 10, such implementations of various aspects of blood vessel tone modulating device embodiments into conventional interventional devices or instruments can be seen.

In FIGS. 8A and 8B a stent retriever type interventional device can be seen being implemented to include a hub with a stimulation circuit 111 in accordance with the various embodiment of the invention, and in this example a plurality of electrodes 77 forming its modulating member 7 can be seen optionally formed at intersections of the struts of its mesh, which is adapted to grab and extract blood clots in blocked arteries.

Some or all of the struts may be used as conducting wires 61, 62 for channeling electrical signals from the stimulation circuit 111 towards the modulating member 7 in order to provide a wave impulse aimed at enhancing tone modulation in a treated blood vessel.

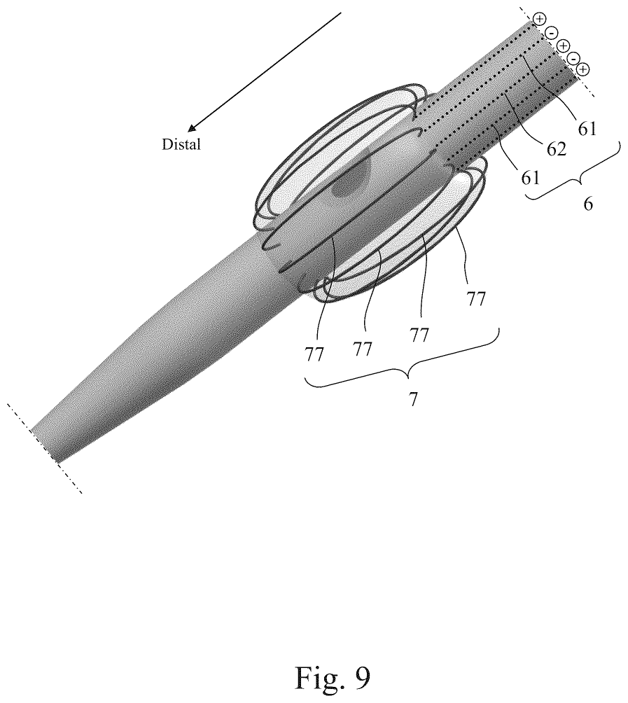

In FIG. 9 a distal region of a balloon type interventional device can be seen being provided with electrodes 77 embedded or formed on its inflatable balloon, at least partially forming the device's modulating member 7 in that location.

Conducting wires 61, 62 channeling electrical signals from a stimulation circuit 111 (such as that see in FIG. 8A) can be seen extending along the catheter's shaft towards here proximal sides of the electrodes 77 in order to communicate electrical signals towards the modulating member 7 to provide a wave impulse aimed at enhancing tone modulation in a treated blood vessel.

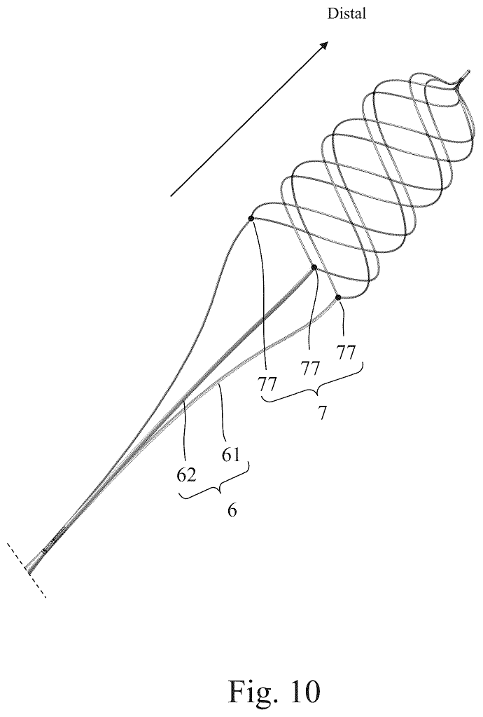

In FIG. 10 an example of distal side of an embolism protection device typically introduced into the treated blood vessel for capturing and removing potentially dangerous debris is shown.

Here, electrodes 77 forming the device's modulating member 7 can be seen optionally formed at the proximal side of the protection device, which is typically widest and most susceptible to contact the treated blood vessel.

Conducting wires 61, 62 channeling electrical signals from a stimulation circuit 111 (such as that see in FIG. 8A) can be seen here too extending along the catheter's shaft towards the electrodes in order to communicate electrical signals towards the modulating member 7 to provide a wave impulse aimed at enhancing tone modulation in a treated blood vessel.

FIG. 2A schematically depicts another optional example of an embodiment of a blood vessel tone modulating device 200 for enhancing tone modulation of blood vessels. Blood vessel tone modulating device 200 includes a connecting hub 201, a supporting structure 8 here embodied as a guide-wire like longitudinal member 203, which is elongated and attached to the hub 201. The distal end of the longitudinal member 203 in this example translates to a tip 204, facilitating the atraumatic distal end. According to some embodiments, longitudinal member 203 is made of metal and/or plastic for needed flexibility for effective blood vessel navigation.

Blood vessel tone modulating device 200 as seen also includes a conductive pathway 6 (see indicated by ‘dashed’ white line in FIG. 2B) that extends along the supporting structure 8 to allow transmission of an electric signals from a stimulation circuit 111 coupled with hub 201 towards a modulating member 7 located along the supporting structure 8, and possibly adjacent the device's tip 204 (see FIG. 2C).

A signal directed to a specific electrode of the modulating member 7 may possibly be performed without substantial interference to other electrodes of the modulating member 7. According to some embodiments, a diameter of the longitudinal member 203 can be in the range of about 0.1 mm to about 30 mm parallel or tapered, and a length in the range of about 3 cm to about 300 cm.

FIG. 2B is a schematic, side view of the proximal end of the tone modulating device of FIG. 2A, according to some embodiments. Hub 201 includes catheter connection interface 214, power button 5, and the stimulation circuit 111. Power button 5 may be designed to facilitate an ergonomic design and allows a user of the device to selectively and on-demand induce an electric signal to modulating member 7.

In principle, the parameters of the components of the stimulation circuit 111 in this and other examples provided herein may be generally similar to those of the aforementioned embodiments already discussed. Moreover, stimulation circuit 111 may be embedded within the discussed hubs of the various embodiments to impart to the various blood vessel tone modulating devices a stand-alone configuration as already discussed hereinabove or may be external and separate from the devices to impart non-stand-alone device embodiments.

FIG. 2C is a schematic, side view of the distal end of the tone modulating device of FIG. 2A, according to some embodiments. The distal end of the longitudinal member 203 may translate to a tip 204, facilitating an atraumatic distal end 242 allowing the progression in the blood vessel anatomy in a safe manner without perforating or dissecting the blood vessel wall. Atraumatic distal end 242 diameter can be in the range of about 0.1 mm to about 30 mm parallel or tapered or twisted or woven.

An electrode 77 of modulating member 7 of device 200 may be exposed at the tip 204 portion to allow inducing electric current to the near or contact tissue. The number of exposed electrodes along the supporting structure 8 and/or tip 204 can be in the range of 1 unit to 100 units. The electrodes 77 can be distributed axially or radially or combined, along supporting structure 8 and/or tip 204.

FIG. 3A schematically depicts yet another optional example of an embodiment of a blood vessel tone modulating device 300 for enhancing tone modulation of blood vessels. Blood vessel tone modulating device 300 includes a connecting hub 301 and a supporting structure 8 in this example in the optional form of a strain relief 302 and a hollow member 303, which is elongated and jointed to a hub 301. The proximal end of the hollow member 303 in this example may be seen optionally supported by the strain relief 302 to the hub in order to provide flexibility that facilitates bending between the hollow member 303 and hub 301 in order to avoid or limit kinks.

The distal end of the hollow member 303 extends towards a tip 304 of the device that may be configured to provide an atraumatic distal end for the device. As seen in FIG. 3C, in this example the blood vessel tone modulating device 300 may be seen embodied as a balloon catheter device including an inflation balloon 305 applied along its supporting structure 8 in this example over its hollow member 303 and connected to an inner lumen of the hollow member to allow balloon inflation. According to some embodiments, hollow member 303 is made of metal and/or plastic for needed flexibility for effective blood vessel navigation.

The blood vessel tone modulating device 300 as seen in FIGS. 3B and 3C also includes a conductive pathway 6 allowing transmission of electrical signals from a stimulation circuit 111 within hub 301 to a modulating member 7 of the device that includes electrodes located along the supporting structure 8, possibly adjacent the device's tip 304 or the balloon's 305 outer surface. Signals may be directed towards a specific electrode, possibly without interference to remaining other electrodes. According to some embodiments, a diameter of hollow member 303 can be in the range of about 0.1 mm to about 30 mm parallel or tapered, and a length in the range of about 3 cm to about 300 cm.

In FIG. 3B is provided a schematic, side view of the proximal end of the tone modulating device of FIG. 3A, according to some embodiments. The hub 301 of the device includes a catheter connection interface 314, a power button 5 and a stimulation circuit 111 such as that previously described herein above.

Power button 5 may be designed to facilitate an ergonomic design for the device and hub and allows a user to selectively and on-demand induce electric signal towards modulating member 7.

Attention is drawn to FIG. 11 schematically showing an endovascular robotic system 1000 for performing minimally invasive procedures in which embodiments of vascular tone modulating devices of the present disclosure may be utilized. The robotic system 1000 can be seen including a robotic manipulator 1001 designed to control a vascular tone modulating device of the present disclosure during an interventional procedure. Such an implementation as seen in FIG. 11 provides an optional example in which embodiments of vascular tone modulating devices may not necessarily be of a standalone type.

Attention is drawn to FIG. 12 schematically showing an interventional device 3000 having a longitudinal axis L exemplifying interventional devices, such as aspiration catheters, embolization catheters, guide catheters, access and sheath catheters, diagnostic catheters (and the like)—that may be configured to include various aspects of the vascular tone modulating device embodiments of the present invention.

A conductive pathway 6 displayed extending along the supporting structure 8 of interventional device 3000 will be further discussed with respect to the enlarged section provided in FIG. 13. It is nevertheless noted that conductive pathway 6 may be implemented on any one of the interventional devices and/or vascular tone modulating devices disclosed herein.

With attention accordingly drawn to FIG. 13 an enlarged section of a braided structural element of conductive pathway 6 is shown. In this example, the displayed braid is in an optional form of threads optionally forming a one over two-under two formation, while noting that other thread formations may equally be applicable.

Threads 65 acting as conductive wires can be seen colored dark gray, while the non-conducting threads 67 remain un-colored. The conductive threads 65 may be formed from metallic material such as tungsten (and the like) and the non-conducting threads 67 may be formed from polymeric fiber material (and in some cases metallic material insulated within a dielectric layer, where such choice may be useful in enhancing structural properties of a supporting structure 8 including such braid if such is useful).

The threads of the braid of this pathway 6 can be seen extending along diagonal directions D1, D2 with respect to the interventional device's longitudinal direction L. The threads extending along different diagonal directions D1, D2 follow opposing helical paths around the interventional device's cylindrical surface and exhibit a true geometric angle of plus and minus “alpha” degrees with respect to axis L on the surface of the device's cylinder (as measured in a flattened view of the cylinder or in cylindrical coordinates).

The structural characteristics of pathway 6 may be affected by various parameters, such as the material of the threads (and the like). One parameter that also may have an effect on the structural characteristics of pathway 6 may be the angle “alpha” defining the diagonal directions D1, D2 along which the threads of the pathway 6 extend. In certain cases and certain angle ranges, a larger angle “alpha” may contribute to the structural characteristic of the pathway 6 e.g. by increasing flexibility, while a smaller angle “alpha” may contribute to the structural characteristic of the pathway 6 e.g. by decreasing flexibility.

Attention is drawn to FIG. 15 illustrating a longitudinal section of a pathway 6 and supporting structure 8 of a vascular tone modulating device, such as the one seen in FIG. 12. In this example, the pathway 6 and supporting structure 8 may be seen including several segments, here three such segments 91, 92, 93—that may be designed to exhibit different structural characteristics.

For example, such segments 91, 92, 93 may differ in the angle “alpha” defining the diagonal directions D1, D2 along which the threads of the pathway 6 extend—in order to impart varying degrees of stiffness/flexibility to the device along its longitudinal extension. In a non-binding example, such a more distal segment may be designed to be more flexible than a more proximal segment in order to enhance device navigation. It is noted that two or more of such segments 91, 92, 93 may be included in a device.

Attention is drawn back to FIG. 13. In this embodiment, the threads running along diagonal direction D1 are arranged in an alternating pattern made out of non-conductive threads 67 and conductive threads 65. As a result, each conductive thread 65 may be provided with an ‘insulation buffer’ by being placed in between non-conductive threads 67, and hence such conductive threads 65 may be ‘bare’ threads that may not necessarily be coated, covered, or enclosed within an insulating material. The threads running along diagonal direction D2 in this example are all non-conductive threads 67.

Typically, ‘bare’ conductive threads 65 running along diagonal direction D1 while being separated one from the other by non-conductive threads 67, may be of opposing polarity. In certain cases (as seen in FIG. 17), several conductive threads 65 (possibly ‘bare’ conductive threads) having a similar polarity may be grouped to extend one alongside the other, while groups with opposing polarity may be separated one from the other by non-conductive threads 67.

The braided pathway 6 (e.g. of FIG. 12, 13, 15 or 17) may be placed as seen e.g. in the enlarged section of FIG. 1B, between inner and outer layers 81, 82 of the supporting structure 8. The inner layer 81 can form an outer boundary of an internal lumen within the interventional device. In one example, the outer layer 82 may be created by positioning a tube made of thermoplastic material over the braid and then applying a reflow process, during which the tube is heated until it becomes pliable or flows to conform to the braid's shape.

As seen in FIGS. 13 and 17, the non-conducting threads 67 appear generally flattened due to the deformation they undergo during the braiding process. To minimize the thickness of the braid and ensure e.g. a snug fit on an interventional device, a flat wire ribbon can be used for the metallic conductive threads 65. This choice provides enhanced flexibility and space-saving advantages compared to conventional round wires. FIG. 14 provides a cross sectional view of such a flat wire ribbon that can be used for a metallic conductive thread 65.

Using flattened conductive threads 65 can also enhance electrical connectivity by providing a larger surface area for contact with the electrodes of the modulating member 7. For instance, flattening a round conductive wire with an outer diameter of approximately 34 microns into a flat wire ribbon having a generally rectangular cross section such as seen in FIG. 14, with a thickness T of about 12 microns, provides a width W being approximately 76 microns for electrode connection. This increased surface area S simplifies the connection process compared to the original 34-micron wire.

Attention is drawn to FIG. 16 schematically showing a cross sectional view perpendicular to a longitudinal axis L of an interventional device such as in FIG. 12 including a braided pathway 6 such as the one seen in FIG. 13.

In an aspect of the present invention, the more robust load-bearing metallic conductive threads 65 may be preferably arranged symmetrically around axis L along the periphery of the interventional device. This symmetrical arrangement enhances the threads' role as part of the device's supporting structure 8, by contributing to its structural symmetry of exhibiting generally uniform resistance to forces applied thereupon. For example, such structural symmetry results in generally uniform resistance to: twisting about the device's axis L, bending in all directions perpendicular to its longitudinal axis of the device (etc.).

What is claimed is:

1. A vascular tone modulating device for controlling the tension and diameter of blood vessels,
the vascular tone modulating device comprising:
a modulating member, a supporting structure, a stimulation circuit and a conductive pathway,
the modulating member comprising at least one electrode,
the supporting structure being suitable for supporting the modulating member within a blood vessel, and
the conductive pathway extending along the supporting structure and electrically connecting the stimulation circuit and the modulating member, wherein the vascular tone modulating device being of a stand-alone configuration.

2. The vascular tone modulating device of claim 1, wherein the stimulation circuit comprising a pulse generator, a battery and a power supply.

3. The vascular tone modulating device of claim 2, wherein the stimulation circuit further comprising an amplifier.

4. The vascular tone modulating of claim 3, wherein the amplifier is any one of: a voltage amplifier, a current amplifier, or a combined voltage and current amplifier.

5. The vascular tone modulating of claim 2, wherein the conductive pathway serving also as a structural component for enhancing structural stability of at least the supporting structure.

6. The vascular tone modulating device of claim 5, wherein the conductive pathway being formed as a braided structural element.

7. The vascular tone modulating device of claim 6, wherein the braided structural element is formed in any one of the following ways: one wire/thread over two-under two; two wires/threads over two-under two; one wire/thread over one-under one.

8. The vascular tone modulating device of claim 7, wherein the conductive pathway comprises one or more electrical conducting wires being interlaced or intertwined together.

9. The vascular tone modulating device of claim 6, wherein the braided structural element having a general cylindrical formation surrounding an internal lumen of the device.

10. The vascular tone modulating device of claim 9, wherein the braided structural element being formed between inner and outer layers, and wherein the inner layer surrounding the internal lumen.

11. The vascular tone modulating device of claim 10, wherein the inner and outer layers being formed from electrical insulating material.

12. The vascular tone modulating device of claim 2, wherein the modulating member comprising a plurality of electrodes.

13. The vascular tone modulating device of claim 12, wherein the conductive pathway comprises a plurality of electrical wires.

14. The vascular tone modulating device of claim 13, wherein the plurality of electrodes of the modulating member being divided into two sets of electrodes, wherein all electrodes of one of the sets being connected to a single electrical wire of the conductive pathway and each one of the electrodes of the other set being connected to a distinct and separate electrical wire of the conductive pathway.

15. The vascular tone modulating device of claim 14, wherein each one of the sets comprises a plurality of electrodes.

16. A method for controlling the tension and diameter of blood vessels comprising the steps of:
providing a vascular tone modulating device having a stand-alone configuration and comprising a modulating member, a supporting structure, a stimulation circuit and a conductive pathway, the modulating member comprising at least one electrode, the supporting structure supporting the modulating member, and the conductive pathway extending along the supporting structure and electrically connecting the stimulation circuit and the modulating member,
inserting at least the supporting structure and the modulating member distally into a blood vessel, and
activating the stimulation circuit to transmit via the conductive pathway electrical signals towards the modulating member in order to create a wave impulse at the modulating member that is delivered towards a vicinity of the blood vessel in order to control the tension and diameter of the blood vessel.

17. The method of claim 16, wherein the stand-alone configuration enables the vascular tone modulating device to operate in an independent manner without the need for external connections or additional equipment.

18. The method of claim 17, wherein the stimulation circuit comprising a pulse generator, a battery and a power supply.

19. The method of claim 18, wherein the stimulation circuit further comprising an amplifier.

20. The method of claim 19, wherein the amplifier is any one of: a voltage amplifier, a current amplifier, or a combined voltage and current amplifier.

21. The method of claim 17, wherein the conductive pathway serving also as a structural component for enhancing structural stability of the supporting structure.

22. The method of claim 21, wherein the conductive pathway being formed as a braided structural element.

23. The method of claim 22, wherein the braided structural element is formed in any one of the following ways: one wire/thread over two-under two; two wires/threads over two-under two; one wire/thread over one-under one.

24. The method of claim 23, wherein the conductive pathway comprises one or more electrical conducting wires being interlaced or intertwined together.

25. The method of claim 24, wherein the braided structural element having a general cylindrical formation surrounding an internal lumen of the device.

26. The method of claim 25, wherein the braided structural element being formed between inner and outer layers, and wherein the inner layer surrounding the internal lumen.

27. The method of claim 26, wherein the inner and outer layers being formed from insulating material.

28. The method of claim 17, wherein the modulating member comprising a plurality of electrodes.

29. The method of claim 28, wherein the conductive pathway comprises a plurality of electrical wires.

30. The method of claim 29, wherein the plurality of electrodes of the modulating member being divided into two sets of electrodes, wherein all electrodes of one of the sets being connected to a single electrical wire of the conductive pathway and each one of the electrodes of the other set being connected to a distinct and separate electrical wire of the conductive pathway.

31. The method of claim 30, wherein each one of the sets comprises a plurality of electrodes.

32. The method of claim 31, wherein the modulating member comprising pairs of electrodes each comprising one electrode from the first one of the sets and another electrode from the second set.

33. The method of claim 32, wherein creating the wave impulse comprises selectively controlling each time only one the pairs to create a wave impulse.

34. The method of claim 33, wherein selectively controlling each time only one the pairs of electrodes to create a wave impulse is by controlling only one of the distinct and separate electrical wires to be connected each time to the device's stimulation circuit.

35. The method of claim 34, wherein the pairs of electrodes are axially spaced apart one from the other along the supporting structure of the device, and the selective controlling of the electrode pairs to create a wave impulse starts from a relative proximal located pair along the supporting structure towards a relative distal located pair along the supporting structure.

36. A vascular tone modulating device for controlling the tension and diameter of blood vessels,
the vascular tone modulating device comprising:
a modulating member, a supporting structure, a stimulation circuit and a conductive pathway,
the modulating member comprising at least one electrode,
the supporting structure being suitable for supporting the modulating member within a blood vessel, and
the conductive pathway extending along the supporting structure and electrically connecting the stimulation circuit and the modulating member, wherein
the conductive pathway serving also as a structural component for enhancing structural stability of the supporting structure.

37. The vascular tone modulating device of claim 36, wherein the conductive pathway being formed as a braided structural element.

38. The vascular tone modulating device of claim 37, wherein the braided structural element is formed in any one of the following ways: one wire/thread over two-under two; two wires/threads over two-under two; one wire/thread over one-under one.

39. The vascular tone modulating device of claim 38, wherein the conductive pathway comprises one or more electrical conducting wires being interlaced or intertwined together.

40. The vascular tone modulating device of claim 39, wherein the braided structural element having a general cylindrical formation surrounding an internal lumen of the device.

41. The vascular tone modulating device of claim 40, wherein the braided structural element being formed between inner and outer layers, and wherein the inner layer surrounding the internal lumen.

42. The vascular tone modulating device of claim 41, wherein the inner and outer layers being formed from electrical insulating material.

43. A catheter for interventional radiology and/or endovascular surgery comprising:
a medical instrument adjacent its distal end for performing a medical procedure within a blood vessel, and a vascular tone modulating configuration for controlling the tension and diameter of the blood vessel,
the vascular tone modulating configuration comprising:
a modulating member, a supporting structure, a stimulation circuit and a conductive pathway,
the stimulation circuit being formed of being part of a hub at a proximal side of the catheter,
the modulating member comprising at least one electrode and being at least partially formed on or with the medical instrument,
the supporting structure being comprised or being part of a portion of the catheter that leads up to the medical instrument and modulating member, and
the conductive pathway extending along the supporting structure and electrically connecting the stimulation circuit and the modulating member, wherein
the vascular tone modulating configuration being of a stand-alone configuration.

44. The catheter of claim 43, wherein the medical instrument being any one of: an inflatable balloon, a stent retriever, an embolism protection device, an aspiration catheter, an embolization catheter, a guide catheter, an access and sheath catheter, a diagnostic catheters (and the like).

45. The catheter of claim 43, wherein the stand-alone configuration enables the vascular tone modulating configuration to control the tension and diameter of the blood vessel in an independent manner without the need for external connections or additional equipment for this purpose.

46. The catheter of claim 43, wherein the stimulation circuit comprising a pulse generator, a battery and a power supply.

47. The catheter of claim 46, wherein the stimulation circuit further comprising an amplifier.

48. The catheter of claim 47, wherein the amplifier is any one of: a voltage amplifier, a current amplifier, or a combined voltage and current amplifier.
